(19) United States
(12) Patent Application Publication
(10) Pub. No.: US 2014/0161733 A1
(43) Pub. Date: Jun. 12, 2014

(54) BIOMARKER-TARGETING CONTRAST AGENTS AND THEIR USE IN MAGNETIC RESONANCE IMAGING FOR DETECTION OF ATHEROSCLEROTIC PLAQUE

(71) Applicant: LUNA INNOVATIONS INCORPORATED, Roanoke, VA (US)

(72) Inventors: Zhiguo Zhou, Winston-Salem, NC (US); Robert P. Lenk, Danville, VA (US); Christopher L. Kepley, Ringgold, VA (US)

(73) Assignee: LUNA INNOVATIONS INCORPORATED, Roanoke, VA (US)

(21) Appl. No.: 14/007,649

(22) PCT Filed: Mar. 29, 2012
(86) PCT No.: PCT/US12/31308

§ 371 Date: Feb. 11, 2014

Related U.S. Application Data
(60) Provisional application No. 61/469,579, filed on Mar. 30, 2011.

(51) Int. Cl. A61K 49/18; A61K 45/00

(52) U.S. Cl. CPC A61K 49/1812; A61K 45/00
USPC 424/9.321; 424/9.323

(57) ABSTRACT

A composition comprising: a liposome having a bilayer structure, a gadofullerene having a high relaxivity, and an amphiphilic receptor ligand. In the composition, the gadofullerene is embedded in the bilayer structure of the liposome. In addition, a method for detecting atherosclerotic plaque in an animal using the composition is described.

### GOVERNMENT LICENSE RIGHTS

The experiments performed in this application were supported in part by Grant No. R43HL087578 awarded by the National Institute of Health. The U.S. Government may therefore have a paid-up license in this invention and may have the right to require patent owner to license others on reasonable terms as provided for by the terms of the above-identified grant.

### BACKGROUND

Targeted imaging is used to reveal the anatomic distribution, size and shape of specific targets in a subject and is currently done primarily with nuclear medicine agents, in which a radioactive tracer is attached to the targeting species. These agents are useful for detecting the distribution of specific targets, but the image resolution is not as good as that of magnetic resonance imaging (MRI). Furthermore, nuclear medicine exposes patients to ionizing radiation, both from the isotope as well as the concomitant CT scans that are required to help orient the image for interpretation.

MR provides very good spatial resolution but does not provide sufficient contrast enhancement to distinguish subtle differences in makeup between diseased vs normal tissues except in unusual circumstances. MRI contrast can be enhanced using T₁ or T₂ contrast agents, and these have expanded the use of MRI for diagnosis of tumors and other diseases which cause breakdown of the circulatory system (e.g., cancer, multiple sclerosis). The enhanced contrast is due to interactions between the water protons and the nuclei of the contrast agent during the image gathering process.

The suitable contrast agents preferably accumulate at specific targets to provide local areas of high contrast, distinct from comparable sites where the agents do not accumulate. There has been some success using targeted T₂ agents, which use magnetic particles such as dextran coated iron oxide nanoparticles. T₁ agents are preferred, but there are several obstacles which must be overcome:
1) paramagnetic T₁ agents, e.g., Gd, are highly toxic,
2) effective contrast enhancement requires the agent accumulate to a relatively high concentration, and
3) the molar concentration of most cellular surface markers which might be useful ligands for targeting is too low to provide sufficient contrast.

The toxicity of Gd can be overcome by enclosing the metal in a chelate, and several different Gd chelates are marketed today. Recently it has been shown that Gd can escape the chelates in vivo, and as a result there is now a black box warning about the dangers associated with the use of chelates in patients whose renal clearance is impaired.

An alternative technology for preventing Gd toxicity is to entrap it inside a carbon nanosphere similar to C₆₀ buckminsterfullerene. The bonds holding these nanospheres together are covalent and resist extreme oxidizing, basic or acidic, conditions. The external surface of fullerenes is pure carbon, so they must be functionalized with hydrophilic groups to make them biocompatible. For example, U.S. Pat. No. 5,717,076 describes a method of functionalizing gadofullerenes via cyclopropanation addition. Also, U.S. Pat. No. 7,358,343 describes metal nitride containing fullerenes functionalized with ligands attached via carbon atoms. It is not obvious how Gd atoms inside carbon nanospheres are able to couple with water protons outside the cage, since the Solomon Bloembergen Morgan equations that govern magnetic interactions predict the distance between the two atoms is too large for sufficient interaction. Recently, the present inventors discovered that functionalizing gadofullerenes with certain polar groups provided relatively high relaxivity using nanoparticles which are 10 nm in diameter or less. This functionalization technology endows the more stable nanospheres with high relaxivity and compatibility with aqueous systems such that they circulate freely and do not aggregate or trigger sequestration in the reticuloendothelial system. See International Application No. WO 2009/054958, published on Apr. 30, 2009 and entitled “Metallofullerene Contrast Agents”.

The high relaxivity functionalization technology on the nanospheres makes them suitable for enhancing contrast, especially within the vasculature. However, further adaptation is required for targeted imaging.

To enhance the contrast of specific targets it is necessary to have the contrast enhancing gadofullerene accumulate at the desired site in sufficient concentration to affect the relaxation time of water protons in the vicinity such that it can be detected during an MRI procedure.

There is an unmet need for MRI contrast agents that will accumulate at specific sites in the body to provide local contrast enhancement during an MRI procedure in the vicinity of specific targets to reveal the anatomic distribution, size and shape of those targets. This ability will improve medical diagnostics by providing earlier detection and more complete anatomical information about diseases.

### SUMMARY

In one embodiment, the invention provides a composition comprising a drug delivery system composed of amphiphilic building blocks, a gadofullerene functionalized with an amine having a C4-C100 alkyl chain and an amine having an alkoxyalkyl chain, and a receptor ligand; wherein the gadofullerene is incorporated in the drug delivery system.

In another embodiment, the invention provides a composition comprising: a liposome drug delivery system having a bilayer structure, a gadofullerene functionalized with an amine having a C4-C100 alkyl chain and an amine having an alkoxyalkyl chain, and an amphiphilic receptor ligand; wherein the gadofullerene is embedded in the bilayer structure of the liposome.

In another embodiment, the invention provides a composition comprising: a liposome having a bilayer structure, a gadofullerene functionalized with an amine having a C1-C20 alkyl chain, and an amphiphilic receptor ligand; wherein the gadofullerene is embedded in the bilayer structure of the liposome.

In some embodiments, the composition also comprises a therapeutic drug; wherein the therapeutic drug is incorporated in the drug delivery system for imaging-guided disease intervention.

In a further embodiment, the invention provides a method for detecting atherosclerotic plaque in an animal, for example a human or a human patient, using the composition. Also provided is a method for simultaneously detecting and treating atherosclerotic plaque in an animal, for example a human or a human patient, and conducting a magnetic resonance imaging to track the disease regression.

### BRIEF DESCRIPTION OF DRAWINGS

FIG. 1. Structures of ATCA1, ATCA2 and ATCA3

FIG. 2. Reaction for Preparation of oxPAPC

FIG. 3. In vitro testing of atherosclerotic-plaque targeting imaging agents for foam cell binding

FIG. 4. ATCA activation of CD36-specific signaling intermediates

FIG. 5. Signal enhancement of atherosclerotic-plaque targeting imaging agents

FIG. 6. Quantification of signal enhancement

FIG. 7. In vivo MRI of atherosclerosis with non-targeted agents

FIG. 8. Signal change of ACTA1 in non-atherosclerotic mice

FIG. 9. Atherosclerotic-plaque targeting imaging agents in major organs

### DETAILED DESCRIPTION OF EMBODIMENTS

The present invention incorporates a high relaxivity gadofullerene compound in a drug delivery system (DDS). The advantage of the DDS is that the delivery system can incorporate 20 to over 1,000 units of contrast enhancing species, which effectively amplifies the amount of signal achieved from each binding event of the ligand with its target. This amplification helps obtain sufficient contrast to be visible during MRI and overcomes the signal density problem described above.

In an embodiment, the drug delivery system is liposomes. Liposomes are spheres made of lipid bilayers. Liposomes (lipid vesicles) are formed when thin lipid films or lipid cakes are hydrated and stacks of liquid crystalline bilayers become fluid and swell. The hydrated lipid sheets detach during agitation and self-close to form large, multilamellar vesicles (MLV) which prevents interaction of water with the hydrocarbon core of the bilayer at the edges. Once these particles have formed, reducing the size of the particle requires energy, for example, sonic energy (sonication) or mechanical energy (extrusion).

The use of liposomes to deliver fullerenes is described. For example, U.S. Pat. No. 7,070,810 describes the synthesis of buckysomes for delivery of drugs from within the lumen. US Patent Application Publication No. 20080213324 describes method for functionalizing fullerenes to enhance their compatibility with phospholipid bilayers. In US Patent Application Publication No. 20080213352, liposome carriers are described with substantially uniform dispersion of fullerenes. Particularly, in Example 9 of US Patent Application Publication No. 20080213352, dodecylaminated gadofullerenes are described as enhancing the loading of fullerenes in liposomes.

To functionalize gadofullerenes and achieve high relaxivity (efficient magnetic coupling), it is necessary to attach ligands to the cage through electronegative atoms. One way to do this is through attaching OH groups, as described in Kato et al., “Lanthanoid Endrohedral Metallofullerenols for MRI Contrast Agents,” J. Am. Chem. Soc., 125(14):4391-97 (2003) and Bolskar et al., “First soluble M@C60 derivatives provide enhanced access to metallofullerenes and permit in vivo evaluation of Gd@C60[C9COOH)2]10 as a MRI contrast agent,” J. Am. Chem. Soc., 125(18):5471-78 (2003). However, the agents described in these publications form large aggregates which are unsuitable for intravenous applications, e.g., Laus et al., “Destroying Gadofullerene Aggregates by Salt Addition in Aqueous Solution of Gd@C₆₀(OH)ₓ and Gd@C₆₀[C(COOH₂)]₁₀,” J. Am. Chem. Soc., 127(26):9368-69 (2005).

The addition chemistry used to attach ligands via electronegative atoms requires harsh conditions, such as strong oxidizing conditions or extreme pH, that damage many fullerene derivatives where the side groups are attached via non polar, e.g., carbon atoms through well-defined addition reactions. Yet, the high relaxivity gadofullerenes synthesized by attaching hydrophilic groups such as short poly(ethylene glycol) groups under these harsh conditions were not compatible with phospholipid bilayers. Even at high molar ratios of lipids to fullerene, the fullerenes did not remain stably associated with liposomes. Dodecyl gadofullerene was prepared by attaching one or more dodecyl hydrocarbon chains using well defined addition chemistry as mentioned above but was unsuccessful. Dodecyl and other alkyl groups-functionalized gadofullerene could be formulated with DDSs that are composed of amphiphilic building blocks such as liposomes, but the product did not have high relaxivity (r₁=6 mM⁻¹sec⁻¹). This low relaxivity was not surprising because the gadofullerene is embedded within the lipid environs of the bilayer, which is relatively inaccessible to water protons. A second possible contribution to the low relaxivity is the low loading capacity for dodecyl gadofullerenes.

Amphiphilic gadofullerenes capable of being efficiently incorporated in drug delivery systems that are composed of amphiphilic building blocks were thus rationally designed for delivery in such DDSs. Amphiphilic building blocks are small molecules, macromolecules and polymers that have at least one hydrophilic moiety and at least one lipophilic moiety, and are capable of self-assembling or co-assembling with other amphiphiles into vesicles or micelles Synthetic and natural lipids such as fatty acids, glycerolipids, phospholipids, sphingolipids, sterol lipids, prenol lipids, saccharolipids, and polyketides are some examples of amphiphilic building blocks. Other examples include block copolymers such as PEGylated polyesters, PEGylated poly(amino acids), and Pluronics, surfactants such as SDS, octanol, and others. The disclosed amphiphilic gadofullerenes are suitable to incorporate in a variety of vesicles, micelles, liposomes, lipid nanoparticles (Solid lipid nanoparticles (SLN) and nanostructured lipid carriers (NLC)), lipid emulsions and hydrogels. Polymeric micelles made of amphiphilic block copolymers such as PLA-PEG and Pluronic PEO-PPO-PEO are capable of delivering such gadofullerenes. In one embodiment, amphiphilic gadofullerenes capable of being efficiently incorporated in lipid bilayers were prepared for delivery in liposomes, and the present inventors were surprised to find that a stable liposome preparation that has high relaxivity (r₁>60 mM⁻¹S⁻¹) could be prepared. In one embodiment, employing a combination of a long-chain alkyl amine with an alkoxyalkyl amine at certain molar ratios during the chemical reactions with pristine gadofullerenes produces compounds that associate with liposomes. In one embodiment, the gadofullerene includes a fullerene with 60-80 carbon atoms. In a further embodiment, the gadofullerene comprises a C80 fullerene. In a preferred embodiment, Gd Trimetasphere® (TMS) is employed. First, the toxic Gd (in cage) is separated from active targeting moieties (outside cage) by the carbon shell. Adding targeting ligands/moieties to the conventional contrast agents may affect the ability of Gd to become free of the compound. Second, the TMS are more sensitive with 3 Gd/molecule. Targeted imaging agents require strong signals by which to report the presence of an agent at a particular location. Third, the fullerene cage can be targeted to disease biomarkers without compromising release of Gd into the body.

The long-chain alkyl includes C4-C100 alkyls. In a further embodiment, the long-chain alkyl amine is a C18 amine. In another embodiment, an alkoxyalkyl amine is a poly(ethylene glycol) functionalized with an alkyl group and an amine group at its two terminals. In a further embodiment, the alkoxyalkyl amine is methyl monoethylene glycol amine (mPEG1-amine) is used. In one embodiment, the molar ratio between the long-chain alkyl amine and the alkoxyalkyl amine is 1:10 to 1:1. These gadofullerenes can be prepared in any suitable conventional method.

In another embodiment, a short-chain alkyl amine is used alone during the chemical reactions with pristine gadofullerenes to produce compounds that can be successfully incorporated in liposomes with high relaxivity (r₁>20 mM⁻¹S⁻¹). This relaxivity is significantly higher than clinically used Gd-chelate MRI agents (r₁ in the range of 3-6 mM⁻¹S⁻¹) or liposomally-formulated Gd-chelate agents (r₁ from 0.4 to 1.6 mM⁻¹S⁻¹). The short-chain alkyl includes C1-C20 alkyls. In a further embodiment, a C4 alkyl amine can be used to produce a gadofullerene compound.

### Stability

The intercalation of the gadofullerene compounds within the bilayers of liposomes is stable. This is confirmed by the fact that extruding the large, plurilamellar vesicles through nucleopore membranes under pressure did not separate the gadofullerenes from the bilayers. Were the association between the lipids and the gadofullerene compound adventitious the mixture would be heterogeneous, with areas rich in phospholipid mixed in with zones rich in gadofullerenes. Subjecting such a mixture to shearing forces would separate the easily deformable lipids from amorphous gadofullerene aggregates. The physical disruption and reformation of bilayers that takes place on extrusion would separate inhomogeneous clusters. Thus, the observed behavior is consistent with a homogeneous dispersion which is stable.

### Relaxivity

Compositions containing the liposomes produced as above have relatively high relaxivity, as measured using a Relaxometer at 0.5 T (Oxford Instruments).

Though not fully understood why the admixture of amines having alkyl chains with, e.g., monoethylene glycol amines during the reaction with gadofullerene yields a product with high relaxivity and yet is stably associated with the liposome carrier, it is believed that there is a delicate balance between the lipophilic moiety and the hydrophilic moiety which allows sufficient flux of water in the vicinity of the gadofullerene to optimize magnetic coupling.

Further, the molar ratios of alkyl amines and mMEG-amine affect the activity of the products. For example, preparations in which the ratio of C18-amine to mMEG-amine is 1:5 appear to be better suited for cellular uptake in tissue culture than preparations where the ratio is 2:5. It is speculated that this difference may be related to the optimum access to bulk water protons to the gadofullerene compounds.

The versatility of this amphiphilic, high relaxivity gadofullerene technology is noteworthy. The same imaging compound can be used in different formulations, which are can be directed towards different targets by incorporating separate ligands within the liposome formulation. In one embodiment, an oxidized phospholipid which binds to the CD36 lipid-scavenging receptors on cell surfaces is incorporated in the gadofullerene/liposome composition. The oxidized CD36 receptor ligand is amphiphilic and anchors to the liposome membrane via its fatty acid chain, exposing the truncated fatty acid/phosphocholine motif which binds to the receptor. Uptake of gadolinium from liposomes formulated with the high relaxivity gadofullerene can occur in cells in tissue culture and contrast enhancement in the ascending aortas of obese mice using MRI have been observed.

It is possible to employ other targeting moieties in the present invention to provide high contrast images of tumors, sites of abnormal inflammation, and other disease conditions.

The amphiphilic high relaxivity nanosphere is enabling technology for targeted imaging, as it fulfills the requirements specified above. The liposome formulation delivers sufficient concentration of imaging agent to the target site to enhance contrast at the site. The use of a drug delivery system provides the further advantage that the targeting moiety can be a constituent of the membrane of the liposome and need not be bound to the imaging module. Thus, the imaging module is adaptable for many targeted imaging products which vary from one another only be the targeting species. That is, the imaging module is the same, the liposome delivery system is the same or similar and the targeting moiety formulated in the bilayers is different.

Atherosclerotic cardiovascular disease results in close to 20 million deaths annually. A hallmark of the disease is the accumulation of lipid plaque in blood vessel walls. This process is initiated when monocytic cells differentiate into macrophage foam cells under conditions with high levels of atherogenic lipoproteins. Vulnerable plaque can dislodge. When dislodged, the plaque enters the blood stream which can result in acute myocardial infarction and stroke. Indeed, a large number of victims of the disease who are apparently healthy die suddenly and without prior symptoms when atherosclerotic plaques dislodge and induce acute myocardial infarction. Clearly, better diagnostic tools are needed to identify incipient disease, monitor disease progression, and pinpoint factors that predict catastrophic ruptures.

At present, physicians cannot specifically detect and quantify plaque buildup in vessel walls. Imaging techniques such as high-resolution magnetic resonance imaging (MRI) is one of several techniques being investigated to identify plaque burden in patients so that interventions can be conducted before rupture occurs.

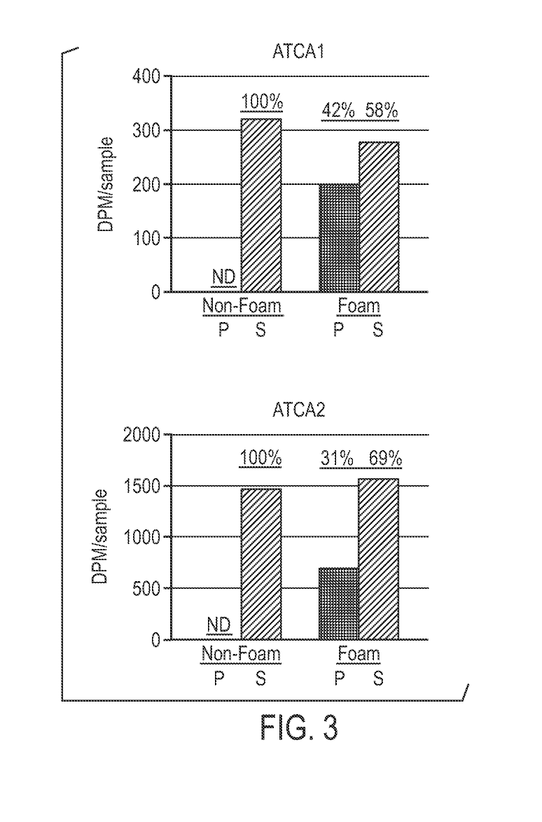

Atherosclerotic plaque contain macrophage foam cells that express CD36 scavenger receptors on their cell surface. these receptors normally and actively internalize the ligands (oxLDL). The CD36 can actively uptake extracellular lipids into their cytoplasmic membrane. As a result, the ATCA contrast agent can be incorporated into foam cells in sufficient quantities that MRI imaging can be performed. To screen for CD36-binding compounds, foam cells from monocytic cell lines and confirmed CD36 expression were induced. As seen in FIG. 3, ATCA1 and ATCA 2 had a significant uptake into the CD36-expressing foam cells. The same compounds as ATCA1 without CD36 ligands was not taken up within the cells suggesting the CD36 receptor was responsible for the uptake of the compounds within the cells. There was no uptake in non-foam cell monocytes or non-monocytic cells (mast cells).

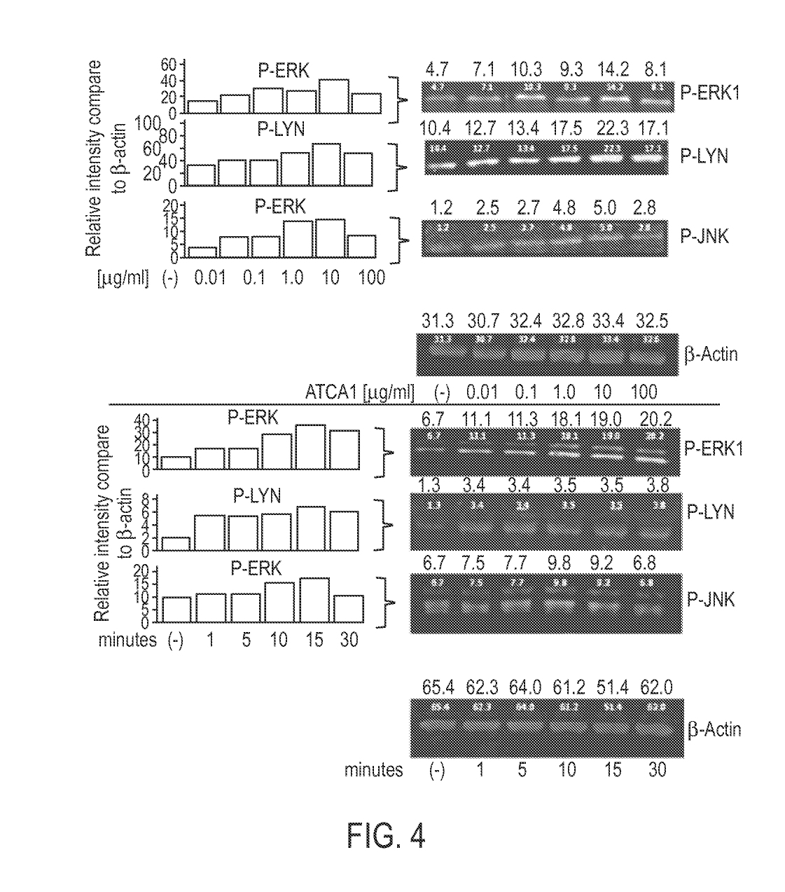

Further, CD36-specific receptor binding of the ATCA was demonstrated by employing Western blotting and quantification of CD36-associated signaling molecules. Previous studies have shown that Erk, Lyn, and JNK2 are activated by the binding of oxLDL to CD36 receptors on macrophages. See Rahaman et al., “A CD36-dependent signaling cascade is necessary for macrophage foam cell formation,” Cell Metab., 4(3):211-21 (2006); Collins et al., “Uptake of oxidized low density lipoprotein by CD36 occurs by an actin-dependent pathway distinct from macropinocytosis,” J. Biol. Chem., 284(44):30288-97 (2009). When foam cells were challenged with ATCA1 there was a dose (FIG. 4A) and time (FIG. 4B) dependent activation of these signaling molecules. This provides further evidence that ATCA specifically target foam cell CD36 receptors through oxLDL binding.

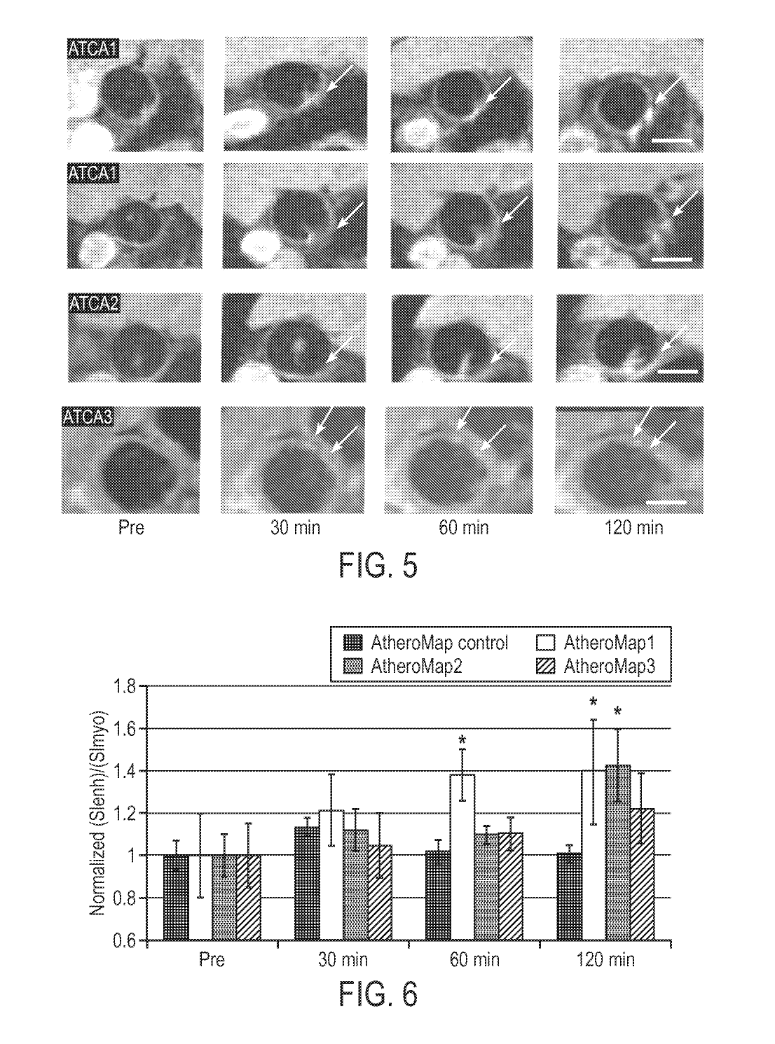

The composition can be used to detect atherosclerotic plaque. It has been described that the lesions in arteries of atherogenic diet-fed ApoE−/− mice progress from fatty streaks to foam cell-containing plaque in a similar way as humans. Kolovuo et al., “Apolipoprotein E knockout models,” Curr. Pharm. Des., 14(4):338-51 (2008); Rosenfeld et al., “Progression and disruption of advanced atherosclerotic plaques in muring models,” Curr. Drug Targets, 9(3):210-16 (2008). Accordingly, APOE−/− mice with atherosclerotic disease were utilized. As can be seen in FIG. 5, mice injected i.v. with the ATCA had a striking enhanced T1 image of the plaque attached in the mouse aorta that could not be seen prior to injection. It is noted that the observation that the imaging agent accumulates over time, suggesting it circulates through the blood for periods long enough for biomarker targeting to occur. Quantification of the image intensity (FIG. 6) demonstrates accumulation of the compounds occurs only after 30 minutes; one and two hour time-points demonstrated the optimal imaging time.

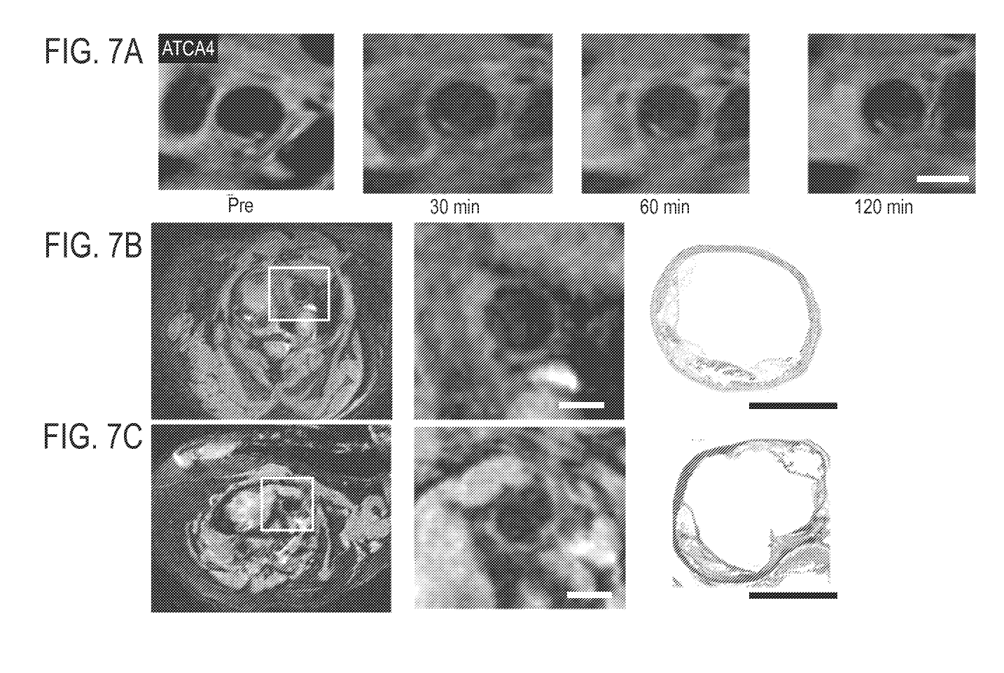

Meanwhile, when control compounds that were essentially the same as ACTA1, ACTA2 and ACTA3 but without the CD36 ligands intercalated within the liposomal membranes were used, as can be seen in FIG. 7A, there was no accumulation in the vessel walls of APOE−/− mice at each of the time points examined. Histochemical evaluation of each animal revealed that plaque accumulation was present (FIGS. 7B and 7C).

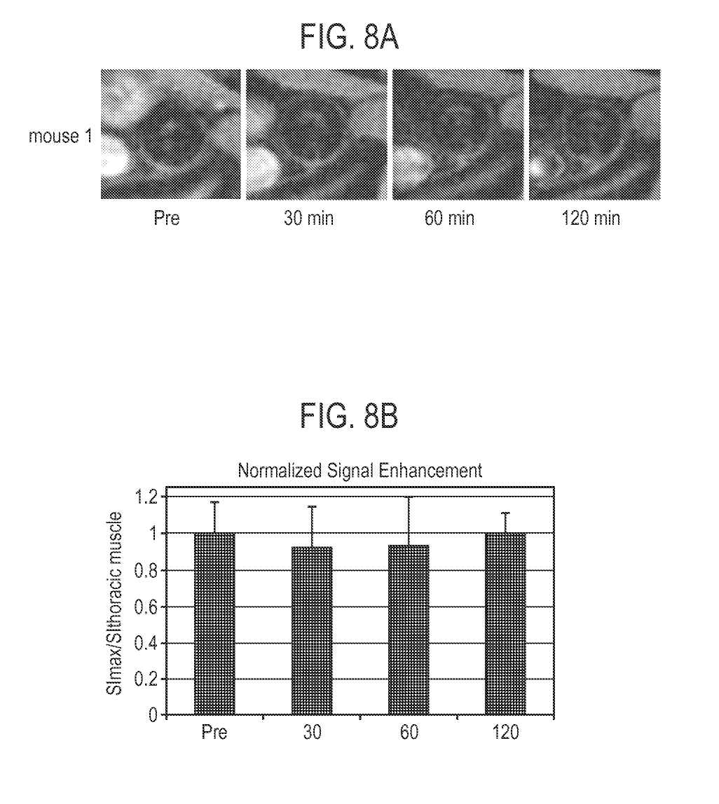

Moreover, separate experiments were performed with the same compounds using non-diseased animals. In these experiments, the plaque-targeting compounds and controls were injected as it was done in FIG. 5 and the descending aorta imaged at the same time points. As can be seen in FIG. 8A, non-diseased, wild-type C57 mice did not demonstrate any signal enhancement in the descending aorta further demonstrating the specificity of the ATCA for plaque detection. These results suggest the ATCA specifically detect disease-induced plaque lesions that are not expressed in normal vessel walls.

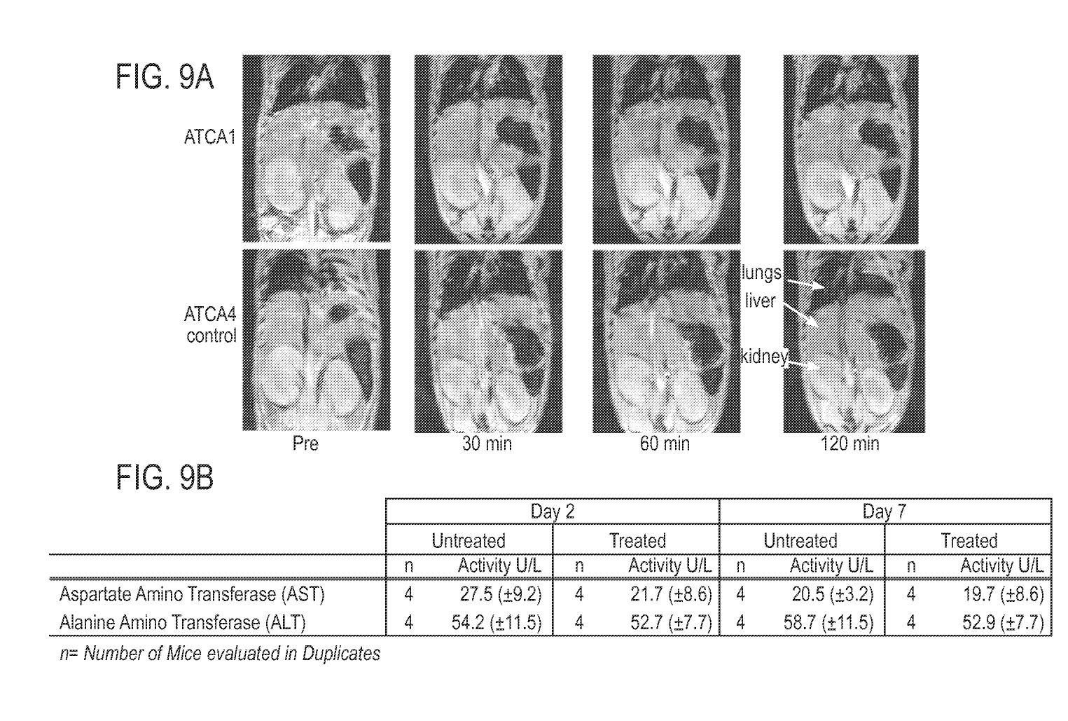

In order to determine the fate of the ATCA, whole body scans after i.v. injection were performed. In all experiments, the injection was well tolerated in all groups of mice with no noticeable adverse reactions observed. There was no accumulation of any of the compounds in any major organs (e.g. liver and kidneys) at the same time points measured in the Apo E−/− mice (FIG. 9A). In separate experiments in which high concentrations of ATCA were injected, there was no significant increase in serum activity of ALT and AST between the untreated and ATCA1-treated animals indicating no liver toxicity (FIG. 9B). Livers from similarly injected mice were examined for Gd accumulation: at day five, one of four mice had detectable Gd (14% of total injected) while at day 14, no mice (n=4) had detectable Gd. Thus, the ATCA appears to be cleared from the body within five days of injection.

The present inventors have developed the novel gadolinium (Gd)-containing C₈₀ fullerenes (Trimetaspheres™, TMS, Gd₃N@C₈₀) that serve as a platform for developing new enhanced MRI contrast agents that target atherosclerotic foam cells. The TMS-based molecules offer 25 fold increased relaxivity compared to other contrast agents, reduced risk of metal toxicity, and can be customized to address issues surrounding solubility, specificity, etc. Further, the plaque-specific biomarkers are employed to develop atherosclerotic targeting contrast agents (ATCA). TMS were functionalized to provide high relaxivity with amphiphilic groups and formulated in liposomes which contained oxidized phospholipids to target the scavenger receptor CD36 found on the surface of macrophage foam cells found in plaque lesions. These contrast agents can specifically bind to and are taken up within foam cells in vitro and are able to detect lesions in plaque-susceptible mice (Apolipoprotein E deficient mice [APO E−/−]). No toxicity was observed using 10 fold concentrations above that optimized for imaging. These results suggest that the ATCA may be a new tool for detecting atherosclerotic plaque. Further, the TMS can serve as a platform for developing biomarker-homing contrast agents for use in diseases that would benefit from imaging quantification with MRI.

Specific non-limiting examples are provided below.

### Example 1

### Synthesis of GdTMS

The TMS was synthesized using an electric-arc process to encapsulate Gd within a carbonaceous cage, C₈₀, according to Stevenson et al., “A stable non-classical metallofullerene family,” Nature, 408(6811):427-28 (2000. The resulting TMS was extracted from the carbon soot, isolated, and purified using HPLC method. The pure TMS was subsequently functionalized with hydrophilic and lipophilic groups, leading to TMS derivatives which magnetically couple with water protons outside the cage and incorporate into liposomes.

The molecular weight of Gd₃N@C₈₀ using matrix-assisted laser desorption/ionization (MALDI) using a time-of-flight (TOF) mass spectrometer was found to be 1,446 kDa. Elemental analysis determined that TMS contains 10.15±0.25 mM Gd which approximates the value estimated from the molecular weight. No free Gd (Gd outside the carbon cage) was detected using an Arsenazo III colorimetric test.

### Example 2

### Synthesis of CD36 Ligands (FIG. 2)

PAPC (1-palmitoyl-2-arachidonyl-sn-glycero-3-phosphocholine or 1-hexadecanoyl-2-eicosatetra-5′,8′,11′,14′-enoyl-sn-glycero-3-phosphocholine) was oxidized by the myeloperoxidase (MPO)-H₂O₂—NO₂— system to generate oxidized PAPC (oxPAPC) which includes HOdiA-PC, KOdiA-PC, HOOA-PC and KOOA-PC species as described in Podrez et al., “Identification of a novel family of oxidized phospholipids that serve as ligands for the macrophage scavenger receptor CD36,” J. Biol. Chem., 277(41):38503-16 (2002). Briefly, 1 mg/mL PAPC solution of small unilamellar vesicles was prepared and oxidized by a mixture of 30 nM MPO, 100 μM glucose and 100 ng/mL glucose oxidase (generating H₂O₂), and 0.5 mM NaNO₂ for 24 hours at 37° C. The reaction was stopped by adding butylated hydroxyltoluene (BHT) and catalase. The oxPAPC lipids were extracted from the oxidized PAPC vesicles with chloroform three times. The combined organic phases were evaporated under nitrogen to dryness. Thin layer chromatography (TLC) was used to demonstrate the successful oxidation of PAPC and to quantify the ratio of oxidized lipids to those non-oxidized. The key binding motif as CD36 ligands was γ-hydroxyl-α,β-unsaturated carbonyl or γ-oxo-α and β-unsaturated carbonyl, where the terminal carbonyl group could be aldehydic or carboxylic. The bioactive and oxidized PAPC lipids were used in the preparation of TMS-encapsulated liposomes.

### Example 3

### Synthesis of 5:1 Amphiphilic GdTMS (5:1 TMS)

The TMS derivatives were synthesized using the amine-butanone peroxide chemistry described in MacFarland et al., “Hydrochalarones: A Novel Endohedral Metallofullerene Platform for Enhancing Magnetic Resonance Imaging Contrast,” J. Med. Chem., 51(13):3681-83 (2008). 20 mg of GdTMS was dissolved in 20 mL ortho-xylene by sonication, and 420 mg 2-methoxy ethylamine (mMEG-amine) and 320 mg octadecylamine (C18-amine, 5:1 molar ratio between mMEG-amine and C18-amine) were subsequently added to the GdTMS solution with vigorous stirring. The mixture was heated in an oil bath with the temperature of 75° C. After all solid materials were dissolved in ortho-xylene, 3.0 mL 2-butanone peroxide solution (35 wt. % in 2,2,4-trimethyl-1,3-pentanediol diisobutyrate) was added, and the mixture was stirred for 60 minutes at 75° C. before it was cooled to room temperature. Volatile solvents such as ortho-xylene were evaporated in vacuo and the residue was loaded onto a silica column for purification. A large volume of ether and THF were used to wash out most of the non-volatile organics. Derivatized GdTMS fractions were collected by eluting the silica column with a mixture of methanol and THF (10%-20% methanol). The major fractions were combined and evaporated to dryness. The product was resuspended in diethyl ether and centrifuged; the top layer solution was decanted. This process was repeated twice to completely remove any non-GdTMS contaminants, such as 2,2,4-trimethyl-1,3-pentanediol diisobutyrate. The isolated product was characterized by FTIR, UV-Vis and NMR which gave the ratio of C18 peak and MEG peak.

### Example 4

### Synthesis of 5:2 Amphiphilic GdTMS (5:2 TMS)

10 mg of GdTMS was dissolved in 10 mL ortho-xylene by sonication, and 200 mg 2-methoxy ethylamine (mMEG-amine) and 320 mg octadecylamine (C18-amine, 5:2 molar ratio between mMEG-amine and C18-amine) were subsequently added to the GdTMS solution with vigorous stirring. The mixture was heated in an oil bath with the temperature of 75° C. After all solid materials were dissolved in ortho-xylene, 1.5 mL 2-butanone peroxide solution (35 wt. % in 2,2,4-trimethyl-1,3-pentanediol diisobutyrate) was added, and the mixture was stirred for 60 minutes at 75° C. before it was cooled to room temperature. Volatile solvents such as ortho-xylene were evaporated in vacuo and the residue was loaded onto a silica column for purification. A large volume of ether and THF were used to wash out most of the non-volatile organics. Derivatized GdTMS fractions were collected by eluting the silica column with a mixture of methanol and THF (10%-20% methanol). The major fractions were combined and evaporated to dryness. The product was resuspended in diethyl ether and centrifuged; the top layer solution was decanted. This process was repeated twice to completely remove any non-GdTMS contaminants, such as 2,2,4-trimethyl-1,3-pentanediol diisobutyrate. The isolated product was characterized by FTIR, UV-Vis and NMR which gave the ratio of C18 peak and MEG peak.

### Example 5

### Synthesis of Butylated GdTMS (C4 TMS)

10 mg of GdTMS was dissolved in 10 mL ortho-xylene by sonication, and 240 mg 1-butylamine was subsequently added to the GdTMS solution with vigorous stirring. The mixture was heated in an oil bath with the temperature of 75° C. After all solid materials were dissolved in ortho-xylene, 1.5 mL 2-butanone peroxide solution (35 wt. % in 2,2,4-trimethyl-1,3-pentanediol diisobutyrate) was added, and the mixture was stirred for 60 minutes at 75° C. before it was cooled to room temperature. Volatile solvents such as ortho-xylene were evaporated in vacuo and the residue was loaded onto a silica column for purification. A large volume of ether and THF were used to wash out most of the non-volatile organics. Derivatized GdTMS fractions were collected by eluting the silica column with a mixture of methanol and THF (10%-20% methanol). The major fractions were combined and evaporated to dryness. The product was resuspended in diethyl ether and centrifuged; the top layer solution was decanted. This process was repeated twice to completely remove any non-GdTMS contaminants, such as 2,2,4-trimethyl-1,3-pentanediol diisobutyrate. The isolated product was characterized by FTIR and UV-Vis.

### Example 6

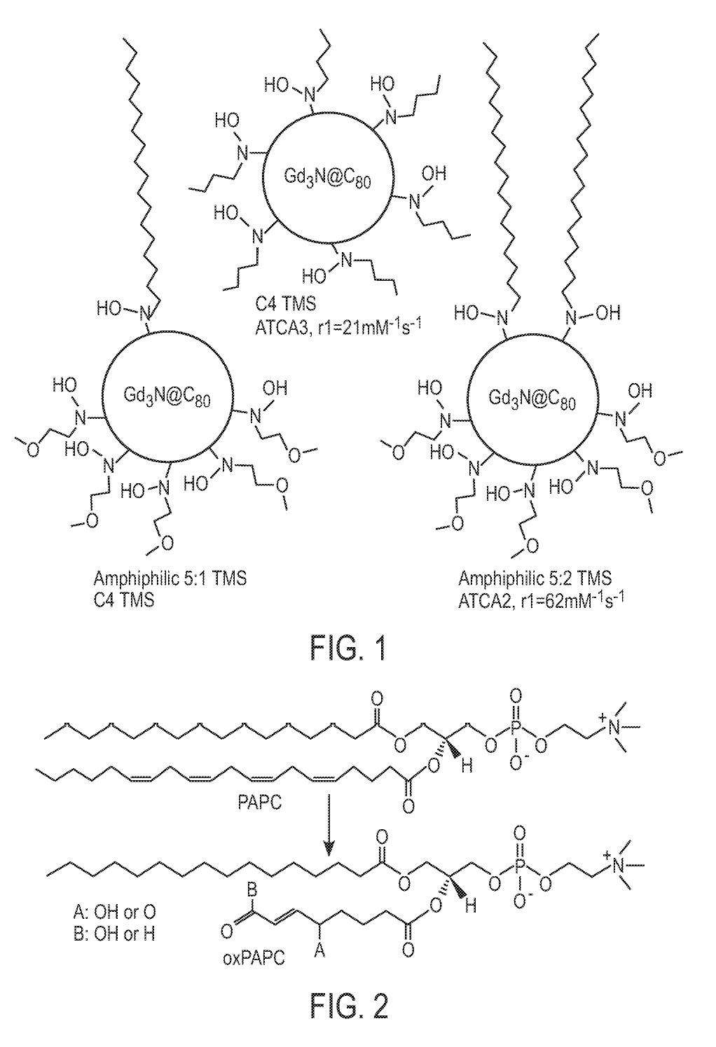

### Liposomal Formulations of CD36-Targeted MRI Contrast Agent (FIG. 1)

ATCA1 was made by mixing 20 parts of regular phosphocholine lipids (DPPC), one part of oxPAPC and five parts of 5:1 amphiphilic TMS in chloroform under nitrogen and the mixture was evaporated to dryness under vacuum to form a thin film on the flask wall. The materials were subsequently hydrated by sonicating the film materials in buffered saline (pH 7.4) using a bath sonicator under nitrogen. The crude liposomes were extruded three times with 400 nm, 200 nm, and 100 nm nucleopore membranes each to produce the final ATCA1 sample as a brownish suspension. The relaxivity of ATCA1 was determined to be 75 mM⁻¹s⁻¹.

ATCA2 and ATCA3 were similarly made starting with the 5:2 amphiphilic TMS and C4 TMS, respectively. The relaxivities of ATCA2 and ATCA3 were determined to be 62 mM⁻¹s⁻¹ and 21 mM⁻¹s⁻¹, respectively.

The control liposome sample ATCA4 has the same ratio of 5:1 TMS and DPPC as ATCA1, but do not contain any oxPAPC.

The incorporation of colored TMS derivatives in liposome bilayers was confirmed by buoyant density test, where the functionalized TMS stayed associated with lipid bilayers on the top of a 40% sucrose cushion under high speed centrifugation conditions that will precipitate any TMS materials if not tightly associated with lipid membranes.

In addition, ATCA samples can be further purified by eluting them on a size exclusion Sephadex column to remove any lipids unincorporated in the liposome bilayer. The co-elution of lipids (DPPC and oxPAPC) with TMS derivatives further demonstrated their tight association in the bilayer structure. Each sample was characterized using dynamic light scattering (DLS) and determined to be around 100±200 nm particles.

The relaxivity r1 of the samples was calculated with experimentally determined Gd concentrations by either an ashing method or ICP-MS and T1 relaxation time of water protons by a benchtop Relaxometer.

### Example 7

### Testing Atherosclerotic-Plaque Targeting Imaging Agent for Foam Cell Binding

The human monocytic cells (U937) (non-foam) were converted into foam cells (foam) using oxLDL and PMA as described in Kuzuya et al., “Oxidation of low-density lipoprotein by copper and iron in phosphate buffer,” Biochim Biophys Acta, 1084(2):198-201 (1991) and Hammad et al., “Oxidized LDL immune complexes induce release of sphingosine kinase in human U937 monocytic cells,” Prostaglandins Other Lipid Mediat., 79(1-2):126-40 (2006). Briefly, monocytic cells were seeded at 10⁶ cells/ml in 24 well plates and treated with or without 0.7 ug/mL phorbol myristilic acid (PMA) and incubated for 24 hours at 37° C., 6% CO₂. Oxidized-LDL (10 ug/mL) was added to the PMA-differentiated macrophage cells and incubated for 48 hours at 37° C., 6% CO₂. Foam cell formation was verified using Oil Red-O (ORO) staining as described in Koopman et al., “Optimisation of oil red O staining permits combination with immunofluorescence and automated quantification of lipids,” Histochem. Cell Bio., 116(1):63-68 (2001). The upregulation of CD36 expression in foam cells was confirmed using both PCR and FACs analysis with CD36-specific antibodies (not shown). As controls, non CD36-expressing cell line 3T3-F442A preadipocytes, and human mast cells were used to detect non-specific binding.

Cells were treated with CD36-targeted or non-targeted controls at various concentrations for 24 hours. Cells were centrifuged and the supernatant saved for Gd analysis. After a quick wash the cell pellet was disrupted by sonication in TES buffer (50 mM Tris, pH 7.4, 1 mM EDTA, and 250 mM sucrose supplemented with 2 μg/ml aprotinin, 1 mM benzamidine, 1 μg/ml pepstatin A, 2 μg/ml leupeptin, 50 μg/ml TPCK, and 0.1 mM PMSF). The cell pellet was subjected to neutron bombardment to determine Gd concentration by measuring disintegrations per minute (DPM) (Biopal Inc). The percentage of Gd in the pellet and supernatant was calculated based on the total amount added to the cells (FIG. 3).

### Example 8

### Western Blotting and Quantification of CD36-Specific Phospho-Signaling Intermediates

To confirm ATCA binding to macrophage foam cell CD36 receptor and examine the mechanisms underlying the interactions between the ATCA and foam cells, Western blotting was performed using a protocol optimized for extracting phospho-proteins from human cells. Foam cells were incubated with or without ATCA1 for various times and concentrations. Following challenge, cells were lysed directly in boiling denaturing sample buffer consisting of tris-buffered saline with triton-X-100 (0.5%) and protease inhibitors. Proteins were separated on a 10% gels in Licor running buffer. Western blotting and image quantification was performed using the Odyssey Imaging System. Band intensities were captured using the Odyssey Imaging System and bands quantified by measuring the number of pixels in each band using a box drawn for the same area of measurement for each separate blot. The band intensity was then normalized for loading by dividing the number of pixels in each band with the housekeeping band intensity (β-actin) performed on the same blot (FIG. 4).

### Example 9

### Atherosclerotic-Plaque Targeting Imaging Agents can Detect Inflammatory Plaque In Vivo

ApoE−/− mice (23 weeks; n=12) fed a lipophilic diet were imaged at the ascending aorta using a 7T MicroMRI Scanner (pre-contrast). The mice were anesthetized initially with isoflurane (3%) and oxygen (3 L/min) in an induction chamber, and were kept under constant sedation via a nose cone. Typical isoflurane percentage and oxygen flow rate during scans were 1.5% and 1 L/min, respectively. Vital statistics were monitored. Plaque-targeting Gd-fullerenes with various ratios of CD36 ligand intercalated within the liposomal membrane (ATCA1, ATCA2, ATCA3) or non-targeted control were injected i.v. (100 μg/100 μl). This concentration was determined to be optimal in initial experiments (not shown).

The mice were placed in supine position, connected to ECG leads, and a respiration pillow. The mice were then positioned with the aorta at the isocenter of the RF coil and the RF coil was positioned in the isocenter of a 7T Bruker BioSpin MRI equipped with a 1000 mT/m gradient set. MR signal transmission and reception was performed with a 35 mm I.D. quadrature RF volume coil. Body temperature was maintained during imaging by blowing thermostatically controlled warm air into the bore of the magnet. After manual shim adjustment, axial and coronal scout images of the ascending abdominal aorta were acquired with a 2D gradient echo sequence with repetition time (TR)=44 milliseconds, echo time (TE)=5 milliseconds, flip angle=30 degrees, slice thickness=1.5 mm and pixel size=234 μm. Then, pre- and post contrast images were acquired at the indicated times using an ECG & respiratory gated Fast Low Angle Shot (FLASH) pulse sequence with parameters:

TR/TE/FA/matrix/FOV/NEX/thk=120 ms/4.9 ms/30 degrees/256/3.0 cm/4/0.38 mm (giving a pixel size of 120 μm). The images are representative of animals from groups of 4 (for each ATCA and control). Scale bar=1.0 mm. The arrows indicate the area of increasing intensity from the MRI contrast agent binding to the plaque (FIG. 5)

A saturation slice was placed over the heart to suppress signal from the flowing blood in the image planes. A fat suppression pulse was applied to reduce chemical shift artifact. After the last post contrast MR acquisition, the animals were sacrificed by CO₂ overdose and cervical dislocation and the abdominal aorta was dissected and excised for histological analysis. All MRI imaging was performed blinded by personnel with no knowledge of targeted and non-targeted compounds.

### Signal Enhancement Measurement

Images acquired after the ATCA injection were examined for areas of hyper-intensity in the aorta wall. Regions appearing hyper-intense after ATCA injection were manually traced using ImageJ (nih.org). The contrast to noise ratio was calculated using a reference region of interest (ROI) in a thoracic muscle. The same regions were traced in the pre-ATCA injection image and in all the post-ATCA time point images. The contrast to noise was calculated using the same reference ROI and then the contrast-to-noise ratio (CNR) values were normalized to the pre-CA CNR value.

### Quantification of Signal Enhancement of Atherosclerotic-Plaque Targeting Imaging Agents

The brightest voxels in the aorta wall that were not bright in the pre-scan images were measured, being careful to exclude voxels that might be part of the low heart rate flow artifact. The mean and std dev of signal intensities in an unenhanced region of the myocardial wall were also measured. The ratio of the Signal Intensity (SI) of the brightest voxel in the aorta wall to the SI of the (non-affected) myocardium for each time point was calculated, making an effort to use voxels in the same area that appeared to become enhanced at later time points, at each time point. The SI-enhanced/SI-myo ratio from each time point to its pre scan SI-enhanced/SI-myo ratio were normalized, making all the pre-scan values 1. Error bars are SEM (N=3). The asterisk (*) indicates statistically significant differences using the student T-test (p<0.04) (FIG. 6).

### Example 10

### A. Non CD36 Targeted Contrast Agents do not Bind Atherosclerotic Plaque In Vivo

As a control, liposome-Gd-fullerene without CD36 ligands (ATCA4) were injected as in Example 8 above and MRI images visualized at the indicated times (FIG. 7A).

### Example 10

### B, C. In Vivo MRI of Atherosclerosis in an ApoE Mouse with Histological Confirmation

The aorta of each mouse was removed and fixed in Tissue-Tek OCT (Miles, Elkhart, Ind.) embedding medium and frozen with liquid nitrogen. The tissue was sectioned onto siloconized slides (Dako) and histopathology assessed using standard hematoxyline stain, as described in Alsaid et al., “Biomimetic MRI Contrast Agent for Imaging of Inflammation in Atherosclerotic Plaque of ApoE−/− Mice: A Pilot Study,” Invest. Radiol., 44(3):151-58 (2009) and Moukdar et al., “Reduced antioxidant capacity and diet-induced atherosclerosis in uncoupling protein-2-deficient mice,” J. Lipid Res., 50(1):59-70 (2009), for assessing plaque accumulation.

An anatomical MR image of aortic area is shown in FIGS. 7B and 7C (left). The same image was magnified to focus on ascending aorta (FIGS. 7B and 7C middle). The mouse was sacrificed and the corresponding histological section demonstrates significant plaque accumulation. It is noted that the atherosclerotic plaque cannot be visualized without first injecting the plaque-targeting contrast agent. Scale bar=1.0 mm.

### Example 10

### D. ATCA do not Nonspecifically Bind to Vessel Walls in Aged-Matched, WT Control Mice

Wild-type, C57/b6 mice (23 weeks) were imaged at the ascending aorta using a 7T MicroMRI Scanner (pre-contrast). ATCA1 was injected i.v. (100 μg/100 μl). Images were acquired at the indicated times (FIG. 8A). The graph in FIG. 8B shows the ratio of aorta wall signal intensity to the thoracic muscle intensity normalized to the pre-scan value showing no change after the injection of targeted contrast agent ATCA1 (n=2).

### Example 11

### Toxicology Evaluation (FIG. 9)

A group of ApoE−/− were injected i.v. with PBS or 1000 μg/100 μl (10 times more than optimized for imaging studies) of ATCA. Mice were sacrificed at Days two, seven, and 14 and alanine aminotranferease (ALT) and aspartate aminotransferase (AST) levels were evaluated in serum. The ALT and AST are transaminase enzymes that leak out into the general circulation when the liver is injured. Data are presented as an average of four (Untreated) or four (Treated) mice ±Standard Deviations. In separate experiments, ATCA was injected as above, livers harvested at Days five and 14, and subjected to neutron bombardment for Gd quantification. An aliquot of the ATCA (not injected) was measured separately to determine the percentage cleared from the mice. No increase in activity was observed between the untreated and treated samples. Activity is measure by Units/L obtained using linear regression from a standard curve.

While the foregoing has been described in detail with reference to specific embodiments thereof, it will be apparent to one skilled in the art that various changes and modifications may be made, and equivalents thereof employed, without departing from the scope of the claims.

All of the above-mentioned references are herein incorporated by reference in their entirety to the same extent as if each individual reference was specifically and individually indicated to be incorporated herein by reference in its entirety.

1. A composition comprising:
a drug delivery system composed of amphiphilic building blocks,
a gadofullerene functionalized with an amine having a C4-C100 alkyl chain and an amine having an alkoxyalkyl chain, and
a receptor ligand;
wherein the gadofullerene is incorporated in the drug delivery system.

2. The composition of claim 1, wherein:
a liposome drug delivery system having a bilayer structure,
a gadofullerene functionalized with an amine having a C4-C100 alkyl chain and an amine having an alkoxyalkyl chain, and
an amphiphilic receptor ligand;
wherein the gadofullerene is embedded in the bilayer structure of the liposome.

3. The composition of claim 2, wherein: (a) the gadofullerene comprises a C60-C80 fullerene; (b) the molar ratio of the C4-C100 alkyl chain and the alkoxyalkyl chain is 1:10 to 1:1; (c) the amphiphilic receptor ligand comprises a CD36 receptor ligand; or (d) the composition has a relaxivity of 20 mM⁻¹s⁻¹ or more.

4. The composition of claim 2, wherein (a) the gadofullerene comprises Gd₃N@C80 fullerene; (b) the C4-C100 alkyl chain comprises a C18 alkyl moiety; (c) the alkoxyalkyl chain comprises methyl monoethylene glycol moiety; or (d) the amphiphilic receptor ligand comprises an oxidized 1-palmitoyl-2-arachidonyl-sn-glycero-3-phosphocholine (oxPAPC).

5. A composition comprising:
a liposome having a bilayer structure,
a gadofullerene functionalized with an amine having a C1-C20 alkyl chain, and
an amphiphilic receptor ligand;
wherein the gadofullerene is embedded in the bilayer structure of the liposome.

6. The composition of claim 5, wherein: (a) the gadofullerene comprises a C60-C80 fullerene; (b) the C1-C20 alkyl chain comprising a C4 alkyl chain; (c) the amphiphilic receptor ligand comprises a CD36 receptor ligand; or (d) the composition has a relaxivity of 20 mM⁻¹s⁻¹ or more.

7. The composition of claim 5, wherein the (a) the gadofullerene comprises Gd₃N@C80 fullerene; or (b) the amphiphilic receptor ligand comprises an oxidized 1-palmitoyl-2-arachidonyl-sn-glycero-3-phosphocholine (oxPAPC).

8. A composition comprising:
the composition of claim 1, and
a therapeutic drug;
wherein the therapeutic drug is incorporated in the drug delivery system for imaging-guided disease intervention.

9. A method for detecting atherosclerotic plaque in an animal comprising administering the composition of claim 1 to a subject in need thereof; and conducting a magnetic resonance imaging.

10. The method of claim 9, wherein said animal is a human.

11. A method for detecting atherosclerotic plaque in an animal comprising administering the composition of claim 5 to a subject in need thereof; and conducting a magnetic resonance imaging.

12. The method of claim 11, wherein said animal is a human.

13. A method for simultaneously detecting and treating atherosclerotic plaque in an animal comprising administering the composition of claim 8 to a subject in need thereof; and conducting a magnetic resonance imaging to track the disease regression.

14. The method of claim 13, wherein said animal is a human.

15. A composition comprising:
the composition of claim 5, and
a therapeutic drug;
wherein the therapeutic drug is incorporated in the drug delivery system for imaging-guided disease intervention.

16. A method for simultaneously detecting and treating atherosclerotic plaque in an animal comprising administering the composition of claim 15 to a subject in need thereof; and conducting a magnetic resonance imaging to track the disease regression.

17. The method of claim 16, wherein said animal is a human.
